(19) United States
(12) Patent Application Publication
(10) Pub. No.: US 2006/0003996 A1
(43) Pub. Date: Jan. 5, 2006

(54) Intralesional treatment of psoriasis

(76) Inventors: Stephen Roth, Gladwyne, PA (US); Robert More, Skillman, NJ (US); Bradford A. Jameson, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/155,450

(22) Filed: Jun. 17, 2005

Related U.S. Application Data
(63) Continuation-in-part of application No. 11/013,969, filed on Dec. 16, 2004.
(63) Continuation of application No. PCT/US03/19595, filed on Jun. 17, 2003.
(60) Provisional application No. 60/414,831, filed on Sep. 27, 2002.
(60) Provisional application No. 60/389,577, filed on Jun. 17, 2002.

(51) Int. Cl. A61K 31/5415

(52) U.S. Cl. 514/228.2

(57) ABSTRACT

The present invention includes the treatment of psoriasis in a human comprising the intralesional administration of a phenothiazine, preferably fluphenazine, to a psoriatic plaque in the patient.

### CROSS-REFERENCE TO RELATED APPLICATIONS

The present application is a continuation-in-part application of U.S. patent application Ser. No. 11/013,969, filed on Dec. 16, 2004, and PCT Application No. PCT/US03/19595, filed on Jun. 17, 2003, each of which claims priority under 35 U.S.C. § 119(e) to U.S. Provisional Patent Application Nos. 60/389,577 and 60/414,831, filed on Jun. 17, 2002 and Sep. 27, 2002, respectively, all of which applications are incorporated herein by reference in their entirety.

### BACKGROUND OF THE INVENTION

Psoriasis is a common human autoimmune condition affecting the skin. Psoriasis can be evident as a small lesion on one area of the body, or or it can affect the majority of the skin surface, the joints and the eyes. Discrete lesions on the skin are typically referred to as psoriatic plaques. The actual cause of psoriasis is unknown, but it is generally accepted that there is a genetic basis as well as environmental basis for the disease. It is known that skin turnover rate in psoriatic skin is much higher than in non-psoriatic skin, the latter occurring about every twenty eight days, while the former occurs in as little as four days. Although various therapies are commercially available for treatment of psoriasis, most treatments require constant application to the affected area and are not without significant side effects.

Serotonin (also referred to as 5-hydroxytryptamine or 5-HT) is a neurotransmitter that has been strongly implicated in the pathophysiology and treatment of a wide variety of neuropsychiatric disorders. Serotonin exerts its effects through a diverse family of serotonin receptor molecules (referred to herein as “5-HT receptors” or “5-HTRs”). Classically, members of the central nervous system (CNS) serotonin receptor family have been grouped into seven (7) subtypes pharmacologically, i.e., according to the effects thereon of various serotonin antagonists. Thus, while all the 5-HT receptors specifically bind with serotonin, they are pharmacologically distinct and are encoded by separate genes. To date, fourteen (14) human CNS serotonin receptors have been identified and sequenced. More particularly, these fourteen separate 5-HT receptors have been grouped into seven (7) pharmacological subtypes, designated 5-HT1, 5-HT2, 5-HT3, 5-HT4, 5-HT5, 5-HT6, and 5-HT7. Several of the subtypes are further subdivided such that the receptors are grouped pharmacologically as follows: 5-HT1A, 5-HT1B, 5-HT1D, 5-HT1E, 5-HT1F, 5-HT2A, 5-HT2B, 5-HT2C, 5-HT3A, 5-HT3B, 5-HT4, 5-HT5A, 5-HT6, 5-HT7. However, when the nucleic and amino acid sequences of the receptors are compared, the percent identity among the subtypes is not correlated to the pharmacological groupings.

Fourteen separate serotonin receptors have been identified encompassing seven subtypes based on, inter alia, structural identity, second messenger system activation, and affinity for certain ligands. Molecular cloning has indicated that 5-HT receptors belong to at least two protein superfamilies: G-protein-associated receptors that have seven putative transmembrane domains (TMDs) (5-HT1A, 1B, 1D, 1E, 5-HT2) and ligand-gated ion channel receptors that have four putative TMDs (5-HT3). The 5-HT2 subfamily is further divided into three classes: 5-HT2A, 5-HT2B, and 5-HT2C. 5-HT2A and 5-HT2C receptor antagonists are thought to be useful in treating depression, anxiety, psychosis, and eating disorders. 5-HT2A and 5-HT2C receptors share about 51% amino acid identity overall and approximately 80% identity in the transmembrane domains. Studies of the 5-HT2A receptor in recombinant mammalian cell lines revealed that the receptor possessed two affinity states, high and low.

Recently, studies have suggested that serotonin may play a role in the immune system because available data demonstrate that serotonin receptors are present on various cells of the immune system. The “mind/body” problem has fascinated people of disparate disciplines for centuries. It has always been understood that there is a link between severe emotions or stress and the immune system. Serotonin is a widely disseminated neurotransmitter and known to play a major role in mood disorders and depression. Its role in modulating the immune response, however, has not been appreciated, much less understood.

There have been reports in the literature about the immunomodulatory effects of adding serotonin exogenously to lymphocyte cultures. Under some circumstances, serotonin has been shown to stimulate the T cells (Foon et al., 1976, J. Immunol. 117:1545-1552; Kut et al., 1992, Immunopharmacol. Immunotoxicol. 14:783-796; Young et al., 1993, Immunology 80:395-400), whereas most laboratories report that high concentrations of added serotonin inhibit proliferation (Slauson et al., 1984, Cell. Immunol. 84:240-252; Khan et al., 1986, Int. Arch. Allergy Appl. Immunol. 81:378-380; Mossner & Lesch, 1998, Brain, Behavior, and Immunity 12:249-271). Thus, the prior art is, at best, unclear as to what role, if any, serotonin might play in modulating the immune response.

With regard to the functional control of the immune response, Gershon et al. (1975, J. Exp. Med. 142:732-738), hypothesized that serotonin was required for mounting a T cell-mediated delayed-type hypersensitivity (DTH) response in mice. However, the authors of this study attributed the dependence of the DTH response on serotonin to the vasoactive properties of this biogenic amine.

A series of studies from the Miles Research Center in West Haven, Conn., showed the presence and involvement of the 5-HT 1a receptors in human and murine T cells (Aune et al., 1990, J. Immnunol. 145:1826-1831; Aune et al., 1993, J. Immunol. 151:1175-1183; Aune et al., 1994, J. Immunol. 153:1826-1831). These studies established that IL-2-stimulated human T cell proliferation could be inhibited by a blockade of tryptophan hydroxylase, i.e., the first enzyme involved in the conversion of tryptophan to serotonin, and that the inhibition could be reversed by the addition of 5-hydroxy tryptophan, i.e., the metabolic product of the inhibited enzyme. Furthermore, they could block human T cell proliferation in vitro with a 5-HT 1a—specific receptor antagonist. In a murine model, they demonstrated that a type 1a receptor antagonist, but not a type 2 receptor antagonist, was able to inhibit the in vivo contact sensitivity response, but not antibody responses, to oxazalone.

Using both type 1a and type 2 receptor antagonist, Laberge et al. (1996, J. Immunol. 156:310-315) serotonin could induce the chemotactic factor, IL-16, from CD8+ T cells and that this activity could be specifically inhibited by the addition of type 2 receptor inhibitors, but not antagonists of the 1a receptor. Thus, although the prior art indicated that serotonin plays a role in the immune system, it was not clear what that role was and there was nothing to suggest that the immune system could be modulated by use of receptor antagonists.

There are a handful of references suggesting that serotonin may play a role the immune response. In 1989, Askenase, and his colleagues demonstrated that a 5-HTR2 antagonist could inhibit a delayed-type hypersensitivity (DTH) response in mice (Amiesen et al., 1989, J. Immunol. 142:3171-3179). Amiesen et al., reasoned that “late-acting DTH effector T cells might express functional 5-HT2R and that these receptors might require in vivo activation in order for the T cells to locally produce the inflammatory lymphokine-dependent aspects of DTH.” These data were subsequently ignored presumably because rodent mast cells contain serotonin but human mast cells do not, such that the results were not applicable to a human immune response. Later, Aune et al. (1994, J. Immunol. 153:489-498), demonstrated that a 5-HTR1a antagonist could inhibit a murine DTH response in vivo and showed that inhibition of the enzyme tryptophan hydroxylase (the first enzyme involved in the conversion of tryptophan to serotonin) could inhibit T cell proliferation. Again, these authors provided important pieces of information, but failed to recognize the larger role of serotonin in the mounting of a T cell-dependent response.

The first evidence that macrophages and lymphocytes expressed receptors capable of responding to serotonin was presented in 1984 (Roszman et al., 1984, Soc. Neurosci. 10:726). Over the intervening years, it has been shown that of the fourteen known pharmacologically distinct serotonin receptors, resting lymphocytes express receptors similar to 5-HT2A, 2B, 2C, 6, and 7 (Ameisen et al., 1989, J. Immunol. 142:3171-3179; Stefulj et al., 2000, Brain, Behavior, and Immunity 14:219-224) and that the 5-HT1A and 5-HT3 receptors are up-regulated upon activation (Aune et al., 1993, J. Immunol. 151:1175-1183; Meyniel et al., 1997, Immunol. Lett. 55:151-160; Stefulj et al., 2000, Brain, Behavior, and Immunity 14:219-224).

Although the functional role of serotonin receptors on lymphocytes and in immune regulation if any has never been defined, it is generally known that serotonin receptors, with the exception of type 3 receptors, which are cation channels, are G-coupled receptors comprising seven transmembrane domains (for a review see Barnes and Sharp, 1999, NeuroPharm. 38:1083-1152). More specifically, the type 1 receptors act on adenylate cyclase, resulting in a down-regulation of cAMP (De Vivo & Maayani, 1986, J. Pharmacol. Exp. Ther. 238:248-252).

In contrast to the 5-HT1A receptors, the 5-HT6 and 5-HT7 receptors, present on resting T cells, act by up-regulating cAMP in response to serotonin (Ruat et al., 1993, Biochem. Biophys. Res. Commun. 193:268-276; Ruat et al., 1993, Proc. Natl. Acad. Sci. USA 90:8547-8551). In an apparently counterintuitive arrangement, the 5-HT6 and 5-HT7 receptors present on the resting cells should act to slow the T cell response, whereas the type la should counteract the signals sent from the 5-HT6 and 5-HT7 receptors. The 5-HT2A and 5-HT2C receptors couple positively to phospholipase C and lead to increased accumulation of inositol phosphates and intracellular Ca²⁺, thereby turning on the protein kinase C signal transduction cascade (for a review see Boess and Martin, 1994, Neuropharmacology 33:275-317).

It was previously hypothesized that serotonin was required for mounting a T cell-mediated delayed-type hypersensitivity (DTH) response in mice (Gershon et al., 1975, J. Exp. Med. 142:732-738). It was concluded that dependence of the DTH response on serotonin was due to the vasoactive properties of this biogenic amine. There have been mixed reports in the literature about the immunomodulatory effects of serotonin. Under some circumstances, exogenous 5-HT has been shown to stimulate activated T cells (Foon et al., 1976, J. Immunol. 117:1545-1552; Kut et al., 1992, Immunopharmacol. Immunotoxicol. 14:783-796; Young et al., 1993, Immunology 80:395-400), whereas most laboratories report that high concentrations of exogenous 5-HT inhibit proliferation of activated T cells (Slauson et al., 1984, Cell. Immunol. 84:240-252; Khan et al., 1986, Int. Arch. Allergy Appl. Immunol. 81:378-380; Mossner & Lesch, 1998, Brain, Behavior, and Immunity 12:249-271). Thus, it is not clear what effect if any serotonin may have on the immune system, since studies suggest that this neurotransmitter both up- and down-regulates the immune response.

There exists a long-felt need to develop therapies for the treatment of psoriasis, wherein the treatment a) does not involve constant application of the drug by the patient; b) has a minimum of side effects; and c) provides symptomatic relief over a long period of time. The present invention meets these needs.

### BRIEF SUMMARY OF THE INVENTION

The invention includes a method of treating psoriasis in a human where the method comprises the intralesional administration of a phenothiazine to a psoriatic lesion on the skin of the patient.

Also included in the invention is a method of treating psoriasis in a human where the method comprises the intralesional administration of fluphenazine, or a derivative thereof, to a psoriatic lesion on the skin of the patient.

In one aspect, the fluphenazine is fluphenazine HCl and in another aspect, the fluphenazine is fluphenazine decanoate.

In one embodiment, the fluphenazine is administered at a dose of between about 2.5 μg and about 50 μg per lesion. In another embodiment, the fluphenazine is administered at a dose of between about 5 μg and about 20 μg per lesion. In yet a further embodiment, the fluphenazine is administered at a dose of between about 7.5 μg and about 15 μg per lesion. In another embodiment, the fluphenazine is administered at a dose of between about 5 μg and about 10 μg per lesion. In a preferred embodiment, the fluphenazine is administered at a dose of about 10 μg per lesion.

In an additional embodiment, the fluphenazine is administered in a volume of between about 0.1 ml and about 2 ml. In another embodiment, the fluphenazine is administered in a volume of between about 0.5 ml and about 1 ml. In yet a further embodiment, the fluphenazine is administered in a volume of between about 0.75 ml and about 1 ml.

In another aspect of the invention, the fluphenazine is injected directly into the lesion. And, in an alternative aspect of the invention, the fluphenazine is administered to the lesion by iontophoresis.

### DETAILED DESCRIPTION OF THE INVENTION

The invention is directed to treatment of the autoimmune disease psoriasis, in a human patient. The treatment regimen includes the administration to the patient an antagonist of the interaction of serotonin, either directly or indirectly, with a serotonin receptor. In one aspect, a phenothiazine compound that is capable of inhibiting, either directly or indirectly, the interaction of serotonin with a serotonin receptor is used. The preferred phenothiazine compound useful in the treatment regimen of the invention is fluphenazine or a derivative thereof. The preferred route of administration of the phenothiazine compound is delivery of the compound directly to the psoriatic lesion, that is, intralesional delivery of the compound to the patient.

Compositions and methods that describe inhibition of the interaction of serotonin with a serotonin receptor are disclosed in U.S. patent application Publication No. 2003/0100570. In addition, the use of fluphenazine and derivatives thereof for modulating the immune response is described in PCT Application No. PCT/US03/19595. Each of these references is incorporated by reference herein in their entirety. In addition, treatment of psoriasis using phenothiazine compounds is suggested in U.S. patent application Publication Nos. US2004/0029860 and US2005/0013853 to Gil-Ad et al. However, these patent applications do not disclose intralesional delivery of the compound. Rather, the disclosure of these references makes clear that phenothiazine compounds administered for treatment of psoriasis should be administered topically to the patient in the form of salves, gels or ointments, wherein the skin of the patient is not punctured in any way. Alternatively, these references disclose parenteral delivery of phenothiazine compounds to a patient for treatment of psoriasis.

In the present invention, it has been discovered that the preferred route for delivery of a phenothiazine compound to a psoriatic patient is direct intralesional delivery of the compound into the psoriatic lesion. This is because a more precise local dose can be administered to the patient which provides a rapid beneficial effect to the patient. In addition, when the phenothiazine compound is administered topically, there is a risk to the patient that a higher than the recommended dose will be administered, potentially resulting in undesirable side effects. This is particularly important if a cream or salve is used and the administration is performed by the patient and not by a trained professional. Similarly, intravenous or intramuscular delivery of a phenothiazine compound to a patient for treatment of psoriasis potentially provides a higher than necessary dose of the compound to the patient, thereby incurring an increased risk of side effects. Intralesional administration has the advantage of being conducted by a trained heathcare professional and ensures therefore that the precise desired dose is administered directly to the affected tissue.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, to “alleviate” a disease means reducing the severity of one or more symptoms of the disease.

By the term “applicator,” as the term is used herein, is meant any device including, but not limited to, a hypodermic syringe, a pipette, an iontophoresis device, and the like, for administering the inhibitor of serotonin interaction with a serotonin receptor to the psoriatic patient.

By the term “effective amount”, as used herein, is meant an amount of an inhibitor that is sufficient to mediate a detectable decrease in transmission of serotonin signaling via a serotonin receptor on a cell. Transmission of a serotonin signal can be assessed using standard methods well-known in the art, such as, but not limited to, those described elsewhere herein, including, for example, assessing the level of binding of serotonin with a receptor and/or assessing the level of activation of a cell.

The skilled artisan would understand that the amount varies and can be readily determined based on a number of factors such as the disease or condition being treated, the age and health and physical condition of the human being treated, the severity of the disease, the particular compound being administered, and the like.

“Identity” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology. “Identity” is used synonymously with “homology” as that term is used in the art.

“Immune response,” as the term is used herein, means a process that results in the activation and/or invocation of an effector function in either the T cells, B cells, natural killer (NK) cells, and/or antigen-presenting cells. Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, and the like.

“Immune cell,” as the term is used herein, means any cell involved in the mounting of an immune response. Such cells include, but are not limited to, T cells, B cells, NK cells, antigen-presenting cells, and the like.

By the term “an inhibitor of the interaction of serotonin with a serotonin receptor,” as used herein, is meant any compound or molecule that detectably inhibits signaling via a serotonin type receptor. Such compounds include a serotonin receptor antagonist, an inverse agonist, and the like.

“Instructional material,” as that term is used herein, includes a publication, a recording, a diagram, or any other medium of expression which can be used to communicate the usefulness of the compound of the invention in the kit for effecting alleviating or treating psoriasis. The instructional material of the kit may, for example, be affixed to a container that contains the compound of the invention or be shipped together with a container which contains the compound. Alternatively, the instructional material may be shipped separately from the container with the intention that the recipient uses the instructional material and the compound cooperatively.

“Intralesional delivery” as used herein, refers to delivery of a compound directly to a psoriatic lesion on the skin. The term includes direct injection of a compound into the lesion, and also includes delivery of a compound to a psoriatic lesion on the skin where the skin is not broken during the delivery (e.g., iontophoresis delivery methods, and the like). However, the term explicitly excludes the use of creams, ointments, salves and gels that are applied directly onto the skin.

A “serotonin receptor” includes a polypeptide that specifically binds with serotonin.

By the term “specifically binds,” as used herein, is meant a receptor which recognizes and binds serotonin family proteins present in a sample (i.e., dopaminergic proteins, adrenergic protein, histamines, melatonin, and serotonin), but does not substantially recognize or bind other molecules in the sample.

To “treat” a disease as the term is used herein, means to reduce the frequency of the disease or disorder reducing the frequency with which a symptom of the one or more symptoms disease or disorder is experienced by an animal.

### DESCRIPTION

The invention relates to methods for treating psoriasis in a human wherein the method comprises direct intralesional delivery of a compound to a patient in need thereof. As noted above, the compound is preferably a phenothiazine and more preferably, fluphenazine or derivatives thereof. However, it is important to emphasize that while the disclosure provided herein recites fluphenazine as the preferred compound, fluphenazine is included herein as an example of a compound useful for treatment of psoriasis by intralesional delivery of the compound to the patient. Thus, the invention should be construed to include derivatives of fluphenazine and other phenothiazines despite the fact that the exemplary compound of the invention is fluphenazine. The invention should also be construed to include any other inhibitor of the interaction of serotonin with its receptor where the inhibitor is delivered intralesionally to the patient.

Pharmaceutical grade fluphenazine HCl suitable for use in this invention may be obtained from a variety of sources, including American Pharmaceutical Partners (Schaumburg, Ill.). In the context of this invention, fluphenazine HCl is intended to include fluphenazine HCl proper, and fluphenazine HCl derivatives, analogs, metabolites, and prodrugs thereof. In particular, fluphenazine decanoate should be specifically construed to be included in the present invention. The effects of fluphenazine decanoate are the same as those of fluphenazine HCl. However, the slow release of the decanoate derivative of fluphenazine from the site of injection results in a prolonged duration of action. Pharmaceutical grade fluphenazine decanoate may be obtained from Bedford Labs (Bedford, Ohio). It is also noted herein that fluphenazine is also known in the art by the names Prolixin™ and Permitil™.

Irrespective of the form of fluphenazine used, the fluphenazine is administered to a patient with psoriasis directly into the psoriatic lesion on the skin of the patient. This is accomplished using a needle and a syringe, or a series of needles and some type of injection or syringe device, where the skin is actually punctured or broken. Alternatively, the fluphenazine is administered to the lesion by iontophoresis, using common iontophoresis technology well known to those of skill in the art. Iontophoresis or ElectroMotive Drug Administration (EMDA) is defined as the topical introduction of ionized drugs into the skin using direct current. Iontophoresis is a very effective method of delivering drugs to the affected site. Instead of injecting the drug directly into the inflamed area, iontophoresis of the drug spreads a high concentration of drug evenly through the tissue. There are several different devices and methods that are useful for iontophoresis of a drug into the skin of a human. For example, ProMed Products, RA Fischer Company, and others, supply devices for iontophoresis of drugs across the skin. In addition, more recent iontophoresis methods and devices are disclosed in U.S. Pat. Nos. RE38,341, RE38,000, RE37,796 and RE36,626 and U.S. patent application No. 2004/0039328 all to Henley, and are hereby incorporated herein by reference in their entirety.

The dose of fluphenazine that is administered to each lesion on the patient is contemplated to be from about 0.1 μg to about 100 μg of fluphenazine. Preferably, the amount of fluphenazine ranges from about 1 μg to about 100 μg of fluphenazine per lesion. More preferably, the amount of fluphenazine administered per lesion is from about 2.5 μg to about 100 μg; even more preferably, from about 5.0 μg to about 100 μg; yet more preferably, from about 7.5 μg to about 75 μg; more preferably, from about 10 μg to about 60 μg; even more preferably, from about 10 μg to about 50 μg; yet more preferably, from about 10 μg to about 40 μg; more preferably, from about 10 μg to about 30 μg; even more preferably, from about 10 μg to about 20 μg, and most preferably, about 10 μg of fluphenazine per lesion. It is understood that smaller increments of amounts of fluphenazine than those recited herein are also included in the invention, provided the fall within the ranges disclosed.

It is preferred that the fluphenazine is administered to the lesion in a volume no greater than about 3 ml, preferably about 2.5 ml, more preferably, about 2.0 ml, yet more preferably, about 1.5 ml, even more preferably, about 1.0 ml and even volumes that are less than 1 ml, including, without limitation, about 750 μl, 500 μl, 250 μl, 100 μl, 75 μl, 50 μl, 25 μl, 10 μl, ≡μl and even as low as about 1 μl. It is further understood that increments in volume that are smaller than those recited herein are included in the present invention, provided they fall within the ranges recited herein.

The skilled artisan, generally the trained physician, will know the frequency with which fluphenazine is administered to any one lesion and to a patient as a whole. For example, the frequency of administration will vary depending upon the severity of the disease, the number and size of the lesions to be treated, the speed at which the lesions heal, the age and gender of the patient, the overall health of the patient, and other factors that are apparent to the skilled artisan.

The specific formulation of fluphenazine used will vary and will also depend any number of factors evident to the skilled artisan. Pharmaceutically acceptable carriers that are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein. In addition, specific pharmaceutical preparations of fluphenazine and its derivatives and other phenothiazines are disclosed in a U.S. patent application entitled “Novel Formulations for Phenothiazines, Including Fluphenazine and Its Derivatives” filed simultaneously herewith, the entire disclosure of which is incorporated herein by reference in its entirety.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for intralesional delivery. Other contemplated formulations include projected nanoparticles, liposomal preparations, resealed erythrocytes containing the active ingredient, and immunologically-based formulations. The compositions may contain pharmaceutically-acceptable carriers and other ingredients known to enhance and facilitate drug administration. Other possible formulations, such as nanoparticles, liposomes, resealed erythrocytes, and immunologically based systems may also be used to deliver the compound to the patient.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. Controlled- or sustained-release formulations of a pharmaceutical composition of the invention may be made using conventional technology.

The invention encompasses various kits relating to intralesional delivery of a phenothiazine compound, e.g., fluphenazine, to a patient with psoriasis. The kit comprises an effective amount of the compound and further comprises an applicator and an instructional material for the use thereof. Although exemplary kits are included herein, the contents of other useful kits will be apparent to the skilled artisan in light of the present disclosure. Each of these kits is included within the invention.

The invention further contemplates the intralesional administration of fluphenazine, or a derivative thereof, to a patient in combination with the administration of a steroid. It is not necessary that the steroid be administered simultaneously with the fluphenazine compound. Rather, the steroid may be administered to the patient before, after or during the administration of fluphenazine and each of these scenarios should be construed to be included in the term “combination” as used herein. The steroid may be administered topically to a lesion on the patient, the steroid may be administered intralesionally as described herein, or the steroid may be administered orally, parenterally, or by any other means used in the art for administration of the steroid in question. Non-limiting examples of steroids useful in the invention include, dexamethazone, tazarotene and methotrexate. Also contemplated is the use non-steroidal compounds, including but not limited to, etrentinate and isotretinoin, for treatment of psoriasis in combination with fluphenazine as disclosed herein.

The disclosures of each and every patent, patent application, and publication cited herein are hereby incorporated herein by reference in their entirety.

Although this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating psoriasis in a human, said method comprising the intralesional administration of a phenothiazine to a psoriatic lesion on the skin of said patient.

2. A method of treating psoriasis in a human, said method comprising the intralesional administration of fluphenazine, or a derivative thereof, to a psoriatic lesion on the skin of said patient.

3. The method of claim 2, wherein said fluphenazine is fluphenazine HCl.

4. The method of claim 2, wherein said fluphenazine is fluphenazine decanoate.

5. The method of claim 2, wherein said fluphenazine is administered at a dose of between about 2.5 μg and about 50 μg per lesion.

6. The method of claim 5, wherein said fluphenazine is administered at a dose of between about 5 μg and about 20 μg per lesion.

7. The method of claim 6, wherein said fluphenazine is administered at a dose of between about 7.5 μg and about 15 μg per lesion.

8. The method of claim 7, wherein said fluphenazine is administered at a dose of between about 5 μg and about 10 μg per lesion.

9. The method of claim 8, wherein said fluphenazine is administered at a dose of about 10 μg per lesion.

11. The method of claim 5, wherein said fluphenazine is administered in a volume of between about 0.1 ml and about 2 ml.

12. The method of claim 11, wherein said fluphenazine is administered in a volume of between about 0.5 ml and about 1 ml.

13. The method of claim 12, wherein said fluphenazine is administered in a volume of between about 0.75 ml and about 1 ml.

14. The method of claim 2, wherein said fluphenazine is injected directly into said lesion.

15. The method of claim 2, wherein said fluphenazine is administered to said lesion by iontophoresis.

16. The method of claim 2, wherein a steroid is administered to said patient in combination with said fluphenazine or a derivative thereof.

17. The method of claim 2, wherein a non-steroidal compound is administered to said patient in combination with said fluphenazine or a derivative thereof.
